(19) United States
(12) Patent Application Publication
(10) Pub. No.: US 2024/0023476 A1
(43) Pub. Date: Jan. 25, 2024

(54) Method for Increasing the Concentration of one or more Bio-actives in Grains

(71) Applicant: The Quaker Oats Company, Chicago, IL (US)

(72) Inventors: El Hadji M. Dioum, Chicago, IL (US); YiFang Chu, Glenview, IL (US)

(21) Appl. No.: 18/223,875

(22) Filed: Jul. 19, 2023

Related U.S. Application Data
(60) Provisional application No. 63/390,752, filed on Jul. 20, 2022.

(51) Int. Cl. A01C 1/02; A61K 36/899; A61K 31/197; A61K 31/196; A23L 7/20; A23B 9/04

(52) U.S. Cl. CPC A01C 1/02; A61K 36/899; A61K 31/197; A61K 31/196; A23L 7/20; A23B 9/04

(57) ABSTRACT

Methods are described for increasing the concentration of one or more bio-actives in grains, and in some instances, the specific bio actives gamma aminobutyric acid (GABA) and/or Avenanthramide (AVA). The increased concentration of the one or more bio-actives is greater than the concentration of the bio-actives in the naturally occurring grains. Compositions that include grains having an increased concentration of one or more bio-actives are described. Methods for promoting health functions such as one or more of reducing oxidative stress, flow mediated dilation (FMD), reducing blood pressure, reducing risk of developing hypertension, improving blood vessel function, improving endothelial function, improving sleep and/or relaxation by providing the compositions are described.

### CROSS-REFERENCE

This Utility Patent application claims the benefit of priority to U.S. Application No. 63/390,752 filed on Jul. 20, 2022, entitled “Method for Increasing the Concentration of One or More Bio-Actives in Grains,” the entire contents of which is incorporated herein by reference.

The present disclosure relates to a method for increasing the concentration of one or more bio-actives in grains, and in some instances, the specific bio actives gamma aminobutyric acid (GABA) and/or Avenanthramide (AVA). The increased concentration of the one or more bio-actives is greater than the concentration of the bio-actives in the naturally occurring grains. The disclosure also relates to grains having an increased concentration of one or more bio-actives and to methods of one or more of reducing oxidative stress, flow mediated dilation (FMD), reducing blood pressure, reducing risk of developing hypertension, improving blood vessel function, improving endothelial function, improving sleep and/or relaxation in mammals, e.g., humans.

### BACKGROUND

Whole grain oats are rich in proteins, dietary fibers, and polyphenols, such as avenanthramides, and provide many potential health benefits to mammals, e.g., humans. For example, naturally germinated grains containing endogenous GABA are sought for their health-promoting phytonutrients because, in part, GABA has been noted as being beneficial for reducing blood pressure, inducing relaxation and enhancing immunity, improving brain function, and postponing intelligence degradation. In plants, GABA is primarily metabolized via a short pathway that has been identified as and named GABA-shunt.

Similarly, Avenanthramides are a group of phenolic alkaloids uniquely found in oats and barley. Avenanthramides have demonstrated antioxidant activities inhibiting fatty acid oxidation based on in vitro cell studies, in vitro total antioxidant capacity and anti-inflammatory activities based on the results of free radicals scavenging and NF-κB activation inhibiting in C2C12 cells. Avenanthramides have also been noted as possessing the potential to help prevent cardiovascular disease based on the evaluation of data on atherosclerosis mouse models since they have similar cholesterol distributions to humans.

More than twenty five (25) types of avenanthramides in oats have been identified, with the most abundant avenanthramides in oats being N-(3′,4′-dihydroxy-(E)-cinnamoyl)-5-hydroxyanthranilic acid (2c), N-(4′-hydroxy-3′-methoxy-(E)-cinnamoyl)-5-hydroxyanthranilic acid (2f), and N-(4′-hydroxy-(E)-cinnamoyl)-5-hydroxyanthranilic acid (2p). It is believed that germination may increase the content of endogenous avenanthramides in oats and barley.

While naturally germinated grains, including oats and barley, have shown an increased GABA and/or avenanthramide content, the increase is variable, not consistent, and oftentimes not efficiently increased. Therefore, there is a need to provide grains such as oats and barley with an increased concentration of one or more bio-actives, and in some instances, with an increased concentration of gamma aminobutyric acid (GABA) and/or Avenanthramide where the increase is greater than that obtained naturally, i.e., where the GABA and/or Avenanthramide content in the grains that have been treated by the described methods is greater than their content without treatment by the described methods.

### SUMMARY

The described methods increase the concentration of one or more bio-actives in grains and in some instances, the bio-actives gamma aminobutyric acid (GABA) and/or Avenanthramide. When describing grains, it is meant to be inclusive of cereal and other grains, including, but not limited to, oats, barley, rice, sorghum, maize, millet, wheat, rye, and may include hulled and hull-less varieties. In some instances, the described methods, compositions, and methods of using the compositions are suitable for oats and barley, For ease of description, unless specifically or explicitly noted otherwise, a mention or reference to oat or oats should be understood to likewise refer to other grains, and particularly to barley.

In one embodiment, the method of increasing the concentration of bio-actives in grains generally includes the sequential steps of optionally hulling the grains (when the grains are hulled), steeping the grains, and then subjecting the steeped grains to a heat shock step followed by a germination step which is terminated by kilning the germinated seed.

Subsequent to kilning, the seeds may be further processed such as by flaking or by milling to produce a flour. The seeds, either further processed or not (which may be referred to as the resulting seeds), may be provided for consumption alone or may be added to an edible product in a form suitable for mammalian consumption. To that end, the resulting seeds may be used for nutrition- and flavor-enhanced drinks, edible food products such as cereals, snacks, crackers, cookies, biscuits, bars, ready-to-eat snack bars, gels, cakes, breads, pasta, other grain-based foods, and the like.

In other aspects, the described methods produce grains having an increased concentration of one or more bio-actives, such as GABA and/or AVA as compared to the concentration of the bio-actives present in seeds that have not been subjected to the described sprouting conditions or subjected to the described methods. In some instances, the increase may be from about 5% to about 50%, i.e., the concentration of the bio-actives in the seeds subjected to the described methods will have a concentration that is between about 105% to about 150% of the concentration of the bio-actives in seeds not subjected to the described methods. In other instances, the concentration of one or more of the bio-actives in the seeds subjected to the described methods will increase from about 5% to 100%, 200%, 300%, 400%, 500%, 600%, 700%, 800%, 900%, 1,000%, or more as compared to seeds that have not been subjected to the described methods (i.e., naturally produced seeds or naturally occurring sprouted seeds).

In this regard, the reference to “increased” is in comparison to the same grains that have not been germinated according to the described methods and which may be referred to as naturally occurring. In other words, the described method provides grains, and in some instances, oats of the species Avena Sativa that have a concentration of GABA and/or Avenanthramides that is higher (greater) than the concentration of GABA and/or Avenanthramides that is present in the naturally occurring grains of the same species. Advantageously, the described method provides oats and/or barley having an increased GABA and/or Avenanthramide concentration without treatment of the oats with enzymes and without exposure of the oats to a fungus.

In some embodiments, compositions effective to modulate energy and mood attributes and methods for modulating such are described. To this end, the compositions contain an effective amount of grains containing an effective amount of bio-actives to modulate one or more of stress reduction, improving sleep and relaxation, reducing oxidative stress, flow mediated dilation (FMD), reducing blood pressure, reducing risk of developing hypertension, improving blood vessel function, and/or improving endothelial function in mammals, i.e., humans.

All percentages used in this description are by weight unless explicitly noted otherwise. Where appropriate given the context, the words “grain”, “grains”, “seed”, and “seeds” may refer to the seeds.

The foregoing aspects and many of the attendant advantages of the present technology will become more readily appreciated by reference to the following Detailed Description, when taken in conjunction with the accompanying simplified drawings of exemplary embodiments. The illustrative, schematic drawings, briefly described here below, are not to scale, are presented for ease of explanation and do not limit the scope of the inventions recited in the accompanying patent claims.

### BRIEF DESCRIPTION OF DRAWINGS

FIG. 1 is an exemplary method for increasing bio-actives in grains.

FIG. 2 is a schematic that illustrates the degree of sprouting and germination of grains.

FIG. 3 is an alternative method for increasing bio-actives in grains.

FIG. 4 shows the concentration of GABA over time of oat seeds that were processed according the described method as compared to oat seeds that were not processed according to the described method.

FIG. 5 shows the concentration of AVA over time of oat seeds that were processed according the described method as compared to oat seeds that were not processed according to the described method.

FIG. 6 shows the percentage increase of GABA under the tested conditions as compared to the low temperature germination method, described below with respect to Example 1.

FIG. 7 shows the GABA content of various heat shock treatments in comparison to the low temperature germination method, described below with respect to Example 1.

FIG. 8 show the GABA content versus the temperature of the oat surface immediately after microwave exposure for various times.

### DETAILED DESCRIPTION

Avenanthramides are polyphenols found in oats. Avenanthramides have been shown to have significant antioxidant activity and have been linked to many beneficial health benefits due to their potent antioxidant activities. In vivo testing has shown that the antioxidant capacity for avenanthramides provides health benefits such as a reduced rate of LDL oxidation thus protecting against cancer and heart disease.

In order to achieve beneficial effects on human health, avenanthramides must be ingested in a sufficient amount. In a 1999 Tufts University study, avenanthramides were confirmed to be bioavailable and remain bioactive in humans after ingestion. After 60 or 120 mg consumption, the maximum concentrations of total plasma avenanthramide were 168 and 560 nM, respectively. A study performed by the University of Minnesota showed that consumption of avenanthramides at doses as low as 0.4 or 9.2 mg/day for 8 weeks increased plasma total antioxidant activity and had dose-response effects on several antioxidant and anti-inflammatory parameters. These effects are probably due to the accumulation and high concentration of avenanthramides in different tissues and organs.

Further, nuclear factor-kappa B (NF-κB) is a family of eukaryotic nuclear transcription factors that regulate the transcription of DNA and are involved in the activation of genes related to inflammatory and immune responses. The regulation of the inflammatory response by NF-κB occurs via the enhancement of the expression of genes encoding pro-inflammatory cytokines, such as tumor necrosis factor (TNF)-α, interleukin (IL)-6, and interleukin (IL)-1β. Activation of NF-κB leads to inflammation that in turn is involved in the pathogenesis of many diseases, such as asthma, rheumatoid arthritis, and inflammatory bowel disease and is at least partially responsible for diseases such as atherosclerosis and Alzheimer's disease. Suppression of NF-κB, a regulator of the immune response to infection, may be useful in limiting the proliferation of cancer cells and reducing the level of inflammation. Studies have shown that avenanthramides inhibit NF-κB activation.

However, the Avenanthramide content in grains varies based on cultivars. Methods that increase avenanthramide content may be useful to help people reach health benefits through regular consumption of oat products

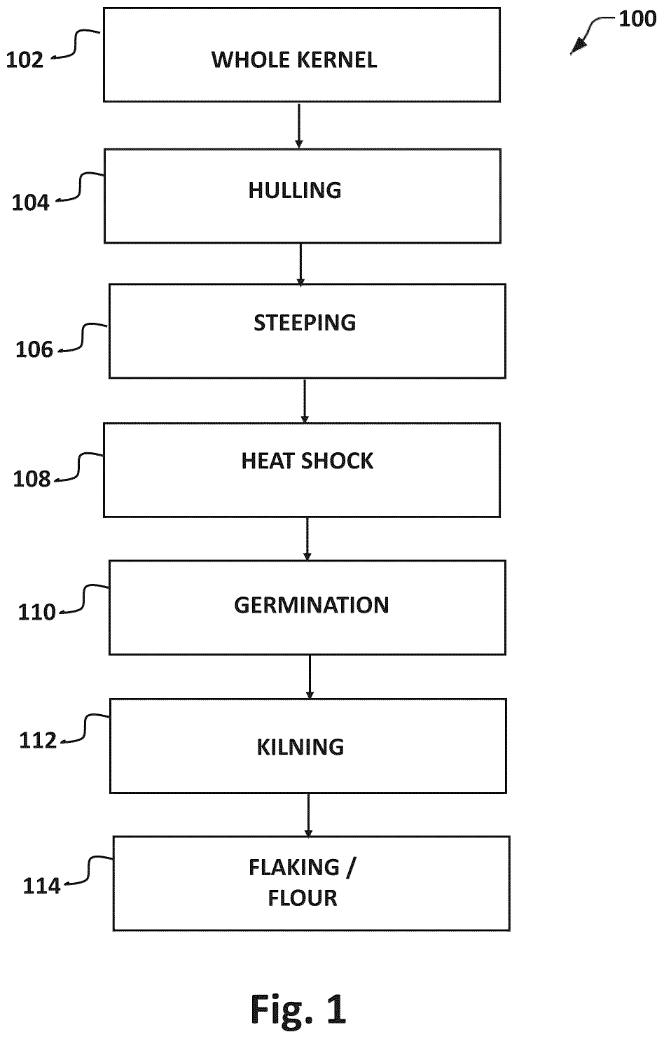

Turning to FIG. 1, a proposed method for increasing the concentration of one or more bio-actives in grains, and in some instances, gamma aminobutyric acid (GABA) and/or Avenanthramide is shown. It will be appreciated that while the proposed method is described in conjunction with oats, the proposed method could be equally applied to other grains, although the skilled artisan will appreciate that one or more steps such as hulling is not required for all grains.

When describing grains, it is meant to be inclusive of cereal and other grains, including, but not limited to, oats, barley, rice, sorghum, maize, millet, wheat, rye, and may include hulled and hull-less varieties. In some instances, the described methods, compositions, and methods of using the compositions are suitable for oats and barley, For ease of description, unless specifically or explicitly noted otherwise, a mention or reference to oat or oats should be understood to likewise refer to other grains, and particularly to barley.

According to one proposed method 100, whole hulled grains or seeds 102 are provided to a hulling step 104. Of course, where the grains or seeds do not have hulls hulling is, of course, not necessary. Prior to providing the whole grains or seeds to the hulling apparatus, the whole grains or seeds may have been passed through one or more sieves or other similar apparatus to remove contaminants based on size. In addition, the whole grains or seeds may optionally have been washed or otherwise cleansed prior to providing them to a hulling step.

Hulling 30 is a known process and any known process for hulling can be used in the present method. For example, hulling may be achieved by compressed air systems, stone hullers, and impact hullers. In an impact huller, the whole grains or seeds are fed through a hollow shaft of the machine to the center of a rotor that is equipped with vanes. The grains or seeds are thrown against an impact ring that is attached to the housing of the machine, the result of which is the release of the hulls from the seeds.

Optionally, the seeds may be sterilized in any known manner. In one instance, the seeds may be sterilized by subjecting them to a solution of NaOCl for a period of time. The solution may be in the range from about 0.05% to about 1% or about 0.1%. The period of time may range from about 1 minute to about 20 minutes and in some aspects may be for about 10 minutes.

After hulling and optional sterilization, the seeds may be subjected to a steeping step 106. In this regard, it is contemplated that the seeds are not pre-treated prior to the steeping step such as by heating the seeds to produce seeds in a state of secondary dormancy. In other words, the hulled seeds are not in a state of secondary dormancy prior to the steeping process.

The steeping process wets the seeds at a given temperature and for a given period of time to provide seeds with a desired moisture content. According to some embodiments, the steeping takes place in an aqueous solution comprising, consisting of, or consisting essentially of water. Typically, the steeping process will occur at a substantially neutral pH or in a pH range from about 6.0 to about 8.0 or from about 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9 or about 8.0.

In some cases the salinity is 50 mM or less and in one embodiment the salinity is as low as measurable, i.e., 0. In some embodiments, the calcium content is about 1% or less and in one embodiment the calcium content is as low as measurable, i.e., 0. The steeping process may be conducted under no-light conditions, i.e., in the absence of light.

The term “moisture content” refers to a proportion of dry weight over hydrated weight. Moisture content may be measured by any method known in the art. By way of example only, an oven, such as a convection, conduction or infrared oven, may be used to determine moisture content. In at least certain exemplary embodiments, a sample may be retrieved and weighed, and placed in the oven. In yet further exemplary embodiments, the sample may be dried, for example with a towel, to remove surface moisture before weighing and/or placing the sample in the oven. Further, the sample may be re-weighed after heating and weight loss calculated to determine moisture loss and moisture content of the sample.

The steeping process may, according to various exemplary embodiments, comprise one or more periods of time where the seeds are exposed to the aqueous solution (referred to interchangeably as “wetting period” or “immersion period”). When more than one wetting period is chosen, it may be desirable to have a period of time between wetting periods where the seeds are optionally aerated.

The wetting period may comprise a step of exposing the seeds to the aqueous solution by any known method. For example, the step may comprise one or more periods of immersing the seeds in the aqueous solution and/or spraying the aqueous solution onto the seeds.

Aeration of the seeds may also be achieved by any known method, such as with the use of fans or compressors, for example. During aeration, the CO₂ produced by the respiring grains may be removed by pulling or pushing the CO₂, for example with fans or compressors, to allow for increased oxygen uptake.

The steeping process may be performed under conditions to produce seeds having a moisture content ranging from about 10% to about 40% by weight. In various embodiments, the steeping process produces seeds having a moisture content ranging, for example, from about 20% to about 38%. In some embodiments, the seeds may have a moisture content of about 10%, 11%, 12%, 13%, 14%, 15%, 16%, 17%, 18%, 19%, 21%, 22%, 23%, 24%, 25%, 26%, 27%, 28%, 29%, 30%, 31%, 32%, 33%, 34%, 35%, 36%, 37%, 38%, 39%, or about 40%.

According to various embodiments, the steeping process may occur over period of time ranging up to about 24 hours, such as about 1 hour to about 24 hours or about 2 hours to about 24 hours. By way of non-limiting example, the steeping process may occur for a period of time ranging from about 4 hours to about 23 hours, such as about 4 hours to about 22 hours, about 4 hours to about 21 hours, about 4 hours to about 20 hours, about 4 hours to about 19 hours, about 4 hours to about 18 hours, about 4 hours to about 17 hours, about 4 hours to about 16 hours, about 4 hours to about 15 hours, about 4 hours to about 14 hours, about 4 hours to about 13 hours, about 4 hours to about 12 hours, about 4 hours to about 11 hours, about 4 hours to about 10 hours, about 4 hours to about 9 hours, about 4 hours to about 8 hours, about 4 hours to about 7 hours, about 4 hours to about 6 hours, or about 6 hours.

In some instances, the steeping process may occur for 1 hour, 2, hours, 3 hours, 4 hours, 5 hours, 6 hours, 7 hours, 8 hours, 9 hour, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours. The skilled artisan will appreciate that the steeping time may depend on the temperature of the aqueous solution, which may depend upon such factors as the facility and ambient conditions such as temperature and humidity. For example, in certain embodiments, the warmer the aqueous solution, the shorter the steeping time, e.g., for the first immersion period. For example, in certain embodiments, the colder the aqueous solution, the longer the steeping time, e.g., for the first immersion period.

According to various exemplary embodiments, the steeping process may comprise a step of wetting the seeds with the aqueous solution for a period of time, optionally followed by a period of aerating the seeds, optionally followed by another step of wetting the seeds with the aqueous solution, and so on. As such, additional aeration and wetting steps may be performed, if desired. By way of non-limiting example only, a first immersion period may range up to about 10 hours, such as about 2 hours to about 10 hours, or about 4 hours to about 6 hours, or about 4 hours or about 6 hours; a first aeration period may range up to about 7 hours, such as about 5 to about 7 hours; and a second immersion period may range up to about 6 hours, such as about 1 to about 6 hours, or about 4 to about 6 hours.

In certain embodiments, the steeping process (wetting and/or aeration) may occur at a temperature ranging from about 10° C. to about 30° C., such as about 10° C. to about 29° C., about 10° C. to about 28° C., about 10° C. to about 27° C., about 10° C. to about 26° C., about 10° C. to about 25° C., about 10° C. to about 24° C., about 10° C. to about 23° C., about 10° C. to about 22° C., about 10° C. to about 21° C., about 10° C. to about 20° C., about 10° C. to about 19° C., about 10° C. to about 18° C., about 10° C. to about 17° C., about 10° C. to about 16° C., about 10° C. to about 15° C., or about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., or about 30° C. In some embodiments, the steeping temperature may be conducted at a temperature of about 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., or about 30° C.

In certain embodiments, the temperature of the aqueous solution may remain relatively constant for a first and a second or subsequent wetting and/or aeration period, although it is not required. It is recognized that the aqueous solution exiting an immersion may be warmer than when the immersion began, as the grains respire. Therefore, any known method for controlling the temperature during wetting and/or aeration may be chosen. For example, additional aqueous solution may be added to the steeping vessel to avoid over-heating the grain and to maintain an ideal temperature of the aqueous solution. In various embodiments, the aeration temperature may be higher or lower than the immersion temperature.

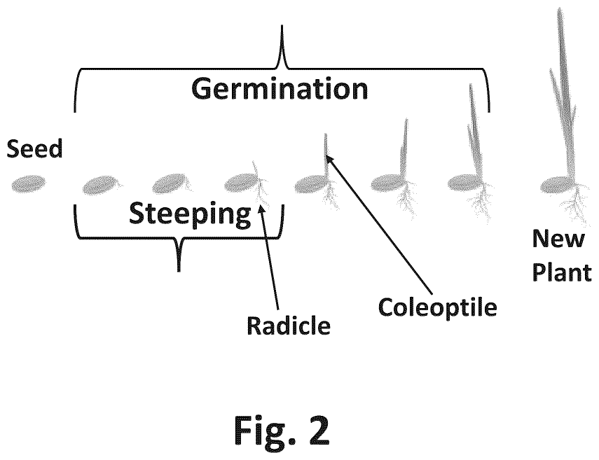

In certain exemplary embodiments, after the steeping process, about 50% to about 100% of the seeds exhibit visible radicle growth and may, in some instances, exhibit the beginning of coleoptile growth. In some embodiments, about 50% to about 75% or about 75% to about 100% of the seeds exhibit visible radicle growth and may, in some instances, exhibit the beginning of coleoptile. Referring to FIG. 2, a schematic representation of seed growth is presented in which the steeping and germination phases are shown.

After completion of the steeping process, the steeped seeds are subjected to a heat shock step or treatment. The heat shock treatment may be conducted using conductive heating or microwave radiation. Conductive heating and microwave radiation apparatuses suitable for performing conductive heating and microwave radiation are well-known and thus, further elaboration of such apparatuses is neither necessary nor warranted.

For conductive heating, the steeped seeds may be subjected to a temperature that is higher than the temperature during steeping and may be in the range of about 25° C. to about 65° C. It is believed that if the heat shock temperature is higher than about 65° C., then the integrity and viability of the seeds would be impacted. In some embodiments, the heat shock temperature may be about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., or about 65° C.

Where the heat shock step is conducted using conductive heating, the heat shock step may be conducted for a period of time from about 0.25, 0.5, 0.75 hours to about 18 hours, or about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, or about 18 hours. In one embodiment the heat shock process step may be conducted for a period of time ranging from about 0.5 hours to about 12 hours. The heat shock treatment may take place in any suitable apparatus in which the temperature and humidity may be controlled.

Where the heat shock treatment step is conducted using microwave radiation, the skilled artisan will appreciate that the time the seeds are subjected to a particular power will vary depending on the power applied to the seeds. For example, it is believed that the period of time the seeds are subjected to a selected microwave power will differ depending on the selected microwave power, i.e., at higher microwave powers, it is expected that the period of time will be shorter as compared to lower microwave powers. With this in mind, the selected microwave power and the period of time can be selected such that water present in the grains is not heated to such an extent to cause such water to steam. In this regard, it is currently believed that if the surface temperature of the seeds during the period of time the seeds are subjected to the microwave radiation is maintained at a temperature greater than about 30° C. and less than about 65° C., the desired increase in bio-active content can be achieved.

With the above in mind, it is desired that the surface temperature of the seeds during a substantial period of time the seeds are subjected to the microwave radiation be maintained at a temperature of about 30° C. or about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., and less than about 65° C.

The microwave radiation may be conducted using any suitable power within a range of about 250 W to about 100 kW. As such, the time period may range from one second (or fraction of one second) at the higher end of the microwave power (i.e., 100 kW) to about 120 seconds at the lower end of the microwave power (i.e., 250 W). With this in mind, it should be appreciated that the time period may range from one second (or fraction of one second) to about 120 seconds, including any value between about 1 and about 120 seconds, i.e., about 10, 20, 30, 40, 50 60, 70, 80, 90, 100, 110, or about 120 seconds.

When the heat shock treatment is complete, the seeds may be germinated under controlled temperature and relative humidity conditions for a defined period of time. In this regard, it has been observed that the bioactive accumulation (i.e., the accumulation of GABA and AVA) during germination starts after the completion of the heat shock period.

In general, the relative humidity is controlled to be about 90%, about 91%, about 92%, about 93%, about 94%, about 95%, about 96%, about 97% or about 98%. The temperature may be controlled at one or more of about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., or about 40° C. In this regard, it has been found, however, that maintaining the temperature at about 43° C. halted the accumulation of one or more bio actives and thus, in general, the germination temperature is typically less than about 43° C.

The defined period of time for germination may be from about 18 hours to about 96 hours, or from about 24 hours to about 72 hours or about 48 hours, In some instances, the defined period of time may be about 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or about 96 hours.

In some instances, the germination temperature may be constant the entire duration of the defined period of time. For example, the germination may be conducted at 24° C. for about 48 hours or about 72 hours. In other instances, the germination temperature may be at a first value for a selected first period of time and then at a second value for a selected second period of time. For example, the germination may be conducted at about 32° C. for about 3 hours and then at about 24° C. for about 54 hours.

In general, it is desired to terminate the germination step before the first leaf emerges. Termination can be accomplished in any suitable manner. In one instance, termination is accomplished by raising the temperature, such as by kilning. In some instances, it is desired to preserve the whole grain status of seed and thus, the germination step may be terminated when the size of the radicle is less than about 90% of the size of the seed, or is less than about 85%, or about 80%, or about 75%, or about 70%, or about 65%, or about 60%, or about 55%, or about 50% of the size of seed. If the radicle is smaller than the size of the grain, the sprouted grain may still be considered a whole grain. In other words, the whole principal anatomical components—the starchy endosperm, germ and bran—are present in the same relative proportions as they exist in the intact caryopsis. Alternatively, the germination step may be terminated when the coleoptile is between about 50% to about 150% of the size of the grain, or between about 50% to about 100% of the size of the grain.

As noted, the steeping and germination steps may be conducted in any suitable apparatus and may, in some instances, be conducted in the same apparatus. As an example, some or all of the steeping and/or germinating steps may be conducted in an apparatus such as that shown in U.S. Pat. No. 8,627,598, the relevant portions of which are incorporated herein by reference.

As noted, kilning terminates the germination step and thus, at the desired time to terminate germination, the seeds are kilned at a defined temperature for a defined period of time to produce seeds having a specified moisture content. The kilning may be conducted in any suitable apparatus where the temperature can be controlled for the defined period of time. In general, kilning may circulate warm air to dry the seeds, help develop flavor and color in the seeds, and arrest the biochemical reactions taking place in the seeds.

During the kilning process, it may be desirable to control air circulation. The phrase “controlled air circulation” and variations thereof, refers to the recirculation of the heated air. In various embodiments, in controlled air circulation, up to about 100% of the air in the kilning vessel is recirculated, such as up to about 65%, to decrease the moisture content of the seeds after steeping (which is from about 29% to about 38% moisture content) to about 2% to about 12% moisture content after kilning. In various embodiments, from about 65% to about 100% of the air is recirculated, and from about 0% to about 35% of the air in the kilning vessel is exhausted. In other various embodiments, from about 75% to about 100% of the air is recirculated, and from about 0% to about 25% of the air in the kilning vessel is exhausted.

For example, the skilled artisan may optimize the convectional removal of the moisture and/or the conductional removal of the moisture. One skilled in the art may, for example, alter fan speeds to create drying air currents, circulation of air, and/or applied temperature to manipulate for the desired end product specification, such as moisture content. Knowledge of kiln operation allows the controller to optimize surface moisture removal, moisture content and grain temperature to meet product specifications.

The kilning process step may range in time up to about 20 hours, such as from about 2 hours to about 18 hours, about 4 hours to about 12 hours, about 6 hours to about 8 hours. In at least one exemplary and non-limiting embodiment, the kilning process lasts about 2, 3, 4, 5, 6, 7, 8, 9, 10, 11, 12, 13, 14, 15, 16, 17, 18, 19, or about 20 hours. Further, the kilning process may be conducted at temperatures ranging from about 45° C. to about 90° C., or about 50° C. to about 85° C., or about 60° C. to about 80° C., or about 65° C. to about 75° C. In some instances, the kilning temperature may be about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or about 90° C.

In various embodiments, the seeds may be kilned for varying times and/or at varying temperatures. For example, the kiln temperature may be stable for a period of time, after which it is increased and then kept stable at the increased temperature for a subsequent period of time, and so on. In certain embodiments, the kiln temperature may remain relatively stable throughout the entire kilning process. In addition, it is not required that the temperature be kept stable at any point during the kilning process; it is possible to increase the temperature, for example gradually, throughout the process. Thus, any combination of time and temperature may be chosen for the kilning process and is done so based on the desired final product specifications, such as the desired moisture content and color.

According to various embodiments, once the kilning process is complete, the seeds may have a final moisture content ranging from about 2% to about 12%, such as about 2%, 3%, 4%, 5%, 6%, 7%, 8%, 9%, 10%, 11%, or about 12%.

Upon completion of the kilning, the grains contain one or more bio actives having a concentration that is greater than grains which have not been subjected to the heat shock process step. In particular the GABA and Avenanthramide content of oats processed according to the method shown in FIG. 1 and described above was greater than the GABA and Avenanthramide content of oats processed according to a conventional method, where there was no heat shock process step.

Also, upon completion of kilning, the seeds may be further processed in a known manner such as by flaking or milling into a flour so that the resulting product may be incorporated with other ingredients for consumption.

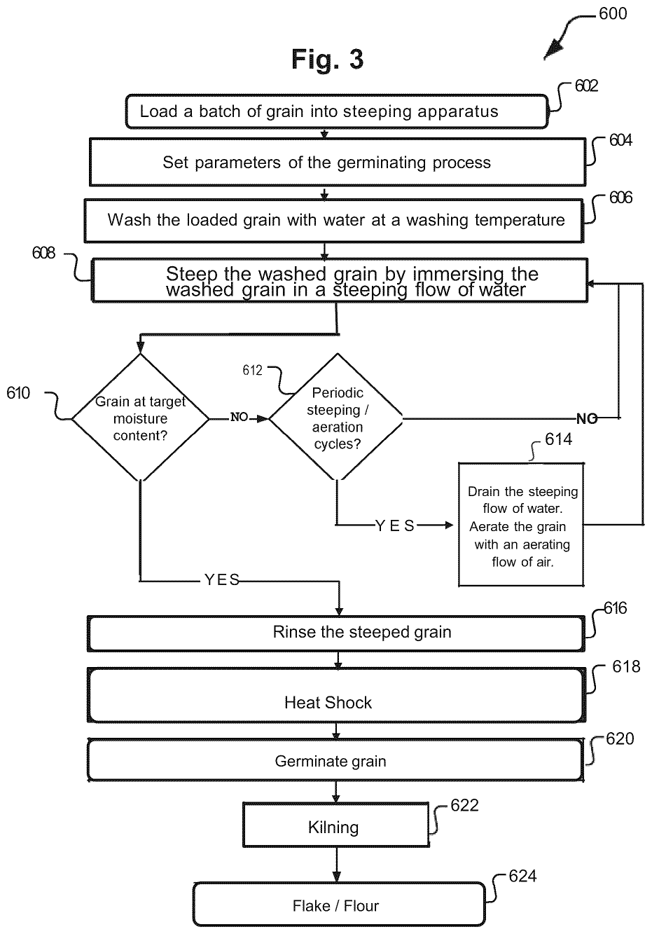

Referring now to FIG. 3, an alternative process 600 is shown for operating a steeping and germination apparatus in an automatic or semi-automatic manner. In at least one embodiment, the process 600 may be implemented by a computer-implemented control system. The process 600 may include loading a batch of grain into a steeping and germination apparatus (not shown). Next, parameters of a steeping process can be set 604. In some cases, setting parameters of a steeping process may include receiving instructions to follow predetermined parameters (e.g., for temperature, cycle step order, cycle step length, and similar parameters). In some cases, setting parameters of the process may include receiving instructions entered by a user.

Next, the process 600 can include washing the loaded grain with water at a washing temperature 606. Washing can generally include immersing the loaded grain in water and/or passing water through the grain in order to remove dirt and debris. In some embodiments, water is pumped and may pass through a dedicated heat exchanger to increase or lower water temperature as required. When it is time to remove water, it may be pumped out and may be directed to a drain or recirculated back through the heat exchanger and returned.

Next, the process 600 may include steeping the washed grains by immersing the washed grains in a steeping flow of water 608. The grains may be fully or partially immersed in the steeping flow of water. In some embodiments, the steeping flow may fully immerse the grains for a predetermined period of time, according to the parameters of the steeping process. As noted above and in some embodiments, the steeping procedure may be interrupted by one or more aeration cycles. The immersing flow of water may be periodically removed, and an aerating flow of air may be passed through the washed grains, before the grains is again immersed.

In some embodiments, the system may determine whether the grains have reached a target moisture content 610. If the grains have not reached target moisture content, the system may continue to immerse the washed grains for an additional length of time 608. In some cases, the system may assess moisture content in conjunction with periodically aerating the grains between steeping cycles 612, in which case the system can drain the steeping flow of water, and aerate the partially steeped grains with an aerating flow of air 614, prior to resuming the steeping process by again immersing the washed grains 608. Target moisture content may be any suitable moisture content, as noted above.

Next, the process 600 can include a heat shock process step 618 using conductive heating or microwave radiation as described above. Thereafter, the grains or seeds are subjected to a germinating stage 620. In some embodiments, the grains or seeds may also be maintained at a predetermined germinating humidity. In some cases, the germinating humidity is approximately 100%, at least 95%, or at least 90%. Aerating the grains or seeds may include passing a flow of air over or through the seeds. The germinating stage may continue for a predetermined period of time, or may continue until the grains or seeds have sprouted. In some cases, the system may determine that the grains or seeds have sprouted by, for example, measuring a carbon dioxide content in the apparatus or in the exhaust stream of air, the carbon dioxide content being indicative of germination in the batch of grain. Alternatively, the germinating stage may be terminated when the size of the radicle is less than about 90% of the size of the seed, or is less than about 85%, or about 80%, or about 75%, or about 70%, or about 65%, or about 60%, or about 55%, or about 50% of the size of seed.

To terminate the germination step, the germinated grains or seeds can be heated such as by being kilned 622. For example, the germinated grains or seeds can be further dried of water content by passing a stream of hot air over and/or through the grains or seeds. In some cases, the germinated grains or seeds can be kilned until they reach a target moisture content. The dried grains or seeds can be subsequently cooled to a handling temperature, e.g., via passing a stream of cooler air through or around the grains or seeds until they can be handled.

Thereafter, the dried and/or cooled sprouted grains may be further processed 624 such as by flaking or milling.

In various embodiments, a computer may manage the system, controlling motors and valves and monitoring sensors to assess the status of the steeping and germinating process. A local area network interface can enable the system to connect to a remote server from which it receives information corresponding to a customized process for each batch of grains while reporting back log data and other operational status. An operator may be able to interact with the computer through a graphical user interface and may be able to perform such functions as view status, and initiate or pause any suitable operational functions.

Embodiments of the system may be operated by, in the computer controller or other hardware or software management module, setting an operating mode including subsets of one or more of the air cycle and water cycle modes described above, in addition to other instructions.

In at least one embodiment, the operator can load the apparatus with a batch of grains and initiate a specific process causing the apparatus to execute a sequence of pre-defined steps. For example, the process 600 illustrates one exemplary sequence, but other sequences of steps are possible. Each step may be interpreted by the machine to activate specific functions and valves, for example one or more of the water cycle modes and air cycle modes, in sequence or in parallel, while monitoring specific sensors and responding conditionally to various events. Events may include, for example: a temperature reaching or exceeding a pre-set range; a pre-set period of time having elapsed; a water level reaching or exceeding a pre-set range; or any other condition. Certain events, such as any which may indicate that the process has strayed outside of desired parameters, may be referred to as fault conditions. The apparatus display and status indicator is maintained and status is reported back to the operations server at selected intervals. Under some conditions the apparatus is paused (or proceeds to pause mode, below) to allow for operator intervention. After all steps have been completed, the apparatus may halt, signaling completion on both the display and status indicator, alerting the operator to unload the finished product from the apparatus.

### Resulting Sprouted Grains or Speeds

As noted above, subsequent to kilning, the seeds may be further processed such as by flaking or by milling to produce a flour. The seeds, either processed or not (may be referred to as the resulting seeds), may be provided for consumption alone or may be added to an edible product in a form suitable for human consumption. To that end the resulting seeds may be used for nutrition- and flavor-enhanced drinks, edible food products such as cereals, snacks, crackers, cookies, biscuits, bars, ready-to-eat snack bars, gels, cakes, breads, pasta, other grain-based foods, and the like.

The described methods produce grains having an increased concentration of one or more bio-actives, such as GABA and AVA as compared to the concentration of the bio-actives present in seeds that have not been subjected to the above-described sprouting conditions or subjected to the described methods. In some instances, the increase may be from about 5% to about 100%, or from about 5%, 10%, 15%, 20%, 30%, 40%, 45%, 50%, 55%, 60%, 65%, 70%, 75%, 80%, 85%, 90%, 95%, or about 100%. In other instances, the concentration of one or more of the bio-actives in the seeds subjected to the described methods will increase from about 5% to 100%, 200%, 300%, 400%, 500%, 600%, 700%, 800%, 900%, 1,000%, or more as compared to seeds that have not been subjected to the described methods (i.e., naturally produced seeds).

In some instances, the concentration of the bio-actives in the seeds subjected to the described methods may have a concentration that is between about 105% to about 200% of the concentration of the bio-actives in seeds not subjected to the described methods. To that end, the concentration may be about 105%, 110%, 115%, 120%, 125%, 130%, 135%, 140%, 145%, 150%, 155%, 160%, 165%, 170%, 175%, 180%, 185%, 190%, 195%, or about 200%. Further, in other instances, the concentration of one or more of the bio-actives in the seeds subjected to the described methods will increase from about 5% to 100%, 200%, 300%, 400%, 500%, 600%, 700%, 800%, 900%, 1,000%, or more as compared to seeds that have not been subjected to the described methods (i.e., naturally produced seeds).

In this regard, reference to “increased” is in comparison to the same grains that have not been steeped, heat shocked, and germinated according to the described methods and which may be referred to as naturally occurring. In other words, the described method provides grains, and in some instances, oats of the species Avena Sativa that have a higher concentration of GABA and Avenanthramides than the concentrations that occur naturally in the species. Advantageously, the described method provides grains having an increased GABA and Avenanthramide concentration without treatment of the grains with enzymes and without exposure of the grains to a fungus.

In some instances, the GABA content in the resulting grains may be in the range of about 100 μg/g seed to about 3,000 μg/g seed. To this end, the GABA content may be about 200 μg/g seed, or about 250, 300, 350, 400, 450, 500, 550, 600, 650, 700, 750, 800, 850, 900, 950, 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900 or about 3,000 μg/g seed.

In some instances the AVA content in the resulting grains may be in the range of about 800 μg/g seed to about 2,500 μg/g seed. To this end, the AVA content may be about 800 μg/g seed, or about 850, 900, 950, 1,000, 1,100, 1,200, 1,300, 1,400, 1,500, 1,600, 1,700, 1,800, 1,900, 2,000, 2,100, 2,200, 2,300, 2,400, or about 2,500 μg/g seed.

### Compositions Containing the Resulting Grains

Sprouting has also been reported to increase other key nutrients of whole grain, including antioxidants, tocopherols, thiamin (B1), riboflavin (B2), pantothenic acid (B5), biotin (B7), folic acid (B9), and fiber. Sprouting of whole grains may also reduce anti-nutrients, such as phytic acid. Phytic acid binds with important minerals such as calcium, magnesium, iron, and zinc and prevents these minerals from being absorbed in the intestine. Phytic acid also chelates niacin making it unavailable for the body. Thus, sprouted whole grains may fill the need to produce whole grains and whole grain food products with desirable taste and nutritional benefits.

The grains prepared according to the described methods may be used in other beverages such as ready-to-drink beverages, fruit juices, dairy beverages, and carbonated soft drinks, and may be added to water, milk, juice, yogurts, puddings, etc. Similarly, the resulting grains may be used in various food products such as bars, cereals, puddings, smoothies, floured beverages, cookies, crackers, gels and the like. The resulting grains may be also be used to make soft food products such as ice cream and soft yogurt. This list is not all-inclusive and one skilled in the art would recognize that the resulting grains may be added to other beverages and food products in accordance with the invention.

A beverage, for example, may contain from about 1% to about 25% of the resulting oats and from about 70% to about 95% total water, typically about 75% to about 90% total water, based on weight of the total drinkable beverage. The balance may contain sweeteners, flavors, fruits and other materials as desired. The water should be suitable for use in food. The total water may be provided in part or in whole from other parts of the drinkable food, especially if milk, juices, or other water containing components are used. For instance, the milk may be dairy (e.g. whole, 2%, 1%, or non-fat) or non-dairy (e.g. soy). The milk may also be produced from powdered milk and water.

The beverage may also include a fruit component. The fruit component can include fruit juice, yogurt containing fruit, fruit puree; fresh fruit, fruit preserves, fruit sorbet, fruit sherbet, dried fruit powder, and combinations thereof. Typically, the fruit component has particles sufficiently small that the component may be safely swallowed without chewing. The fruit component and/or an added acidulant can be adjusted to obtain a desired pH, for example a pH of less than about 4.6.

Additional ingredients may be added to the beverage and food products. Such ingredients may include non-oat or non-grain-based ingredients. For example, flavoring agents, coloring agents, sweeteners, salt, as well as vitamins and minerals can be included. In one embodiment of the invention, flavoring agents such as strawberry, chocolate or cinnamon flavor is added to enhance the taste of the product. Other fruit flavoring agents may also be useful to provide different tastes to the food product, for example, strawberry, mango, banana and mixtures thereof. Spices, in particular, cinnamon, can be used. In addition, any desired flavor or flavors can be used. Suitable sweeteners—artificial or natural can be added in the food product to provide a desired sweetness. For example, brown sugar, maple sugar or fruit sugar can be used. The additional ingredients may be present in a range of about 1 wt. % to 75 wt. % of the total weight of the product.

Other optional ingredients may include, but are not limited to, salt, hydrocolloids, polysaccharides, thickeners, caffeine, dairy, coffee solids, tea solids, herbs, nutraceutical compounds, electrolytes, vitamins, minerals, amino acids, preservatives, alcohol, colorants, emulsifiers, and oils as known in the art.

### Methods of Using Resulting Grains and

### Compositions Containing Resulting Grains

In some embodiments, the described methods provide compositions that are effective to modulate energy and mood attributes in mammals, e.g., humans. To this end, the described methods provide compositions containing the resulting grains (resulting seeds) with effective amounts of bio-actives to modulate one or more of stress reduction, improving sleep and relaxation, reducing oxidative stress, flow mediated dilation (FMD), reducing blood pressure, reducing risk of developing hypertension, improving blood vessel function, and/or improving endothelial function.

The compositions may comprise, consist essentially of, or consist of the resulting grains. In alternative embodiments, the composition may be formed as a food product containing the resulting grains, as described above. With this mind, it is contemplated to provide an amount of the resulting grains such that the composition contains GABA in amounts from about 20 mg/40 g serving and up to about 120 mg/40 g serving to enhance relaxation, improve sleep and/or relaxation, reduce blood pressure, improve cardiovascular health, reduce stress, reduce oxidative stress, flow mediated dilation (FMD), reduce risk of developing hypertension, improve blood vessel function, and/or improve endothelial function.

To that end, a method of enhancing or improving a feeling of relaxation includes providing a composition containing the resulting grains to provide GABA in amounts between about 20 mg/40 g serving to about 40 mg/40 g serving or from about 20, 21, 22, 23, 24, 25, 26, 27 28, 29, 30, 31, 32, 33, 34, 35, 36, 37, 38, 39, or about 40 mg/40 g serving. In other embodiments, a method of enhancing a feeling of sleepiness or decreasing the time duration to sleep includes providing a composition containing the resulting grains to provide GABA in amounts between about 50 mg/40 g serving to about 150 mg/40 g serving or from about 50, 55, 60, 65, 70, 75, 80, 85, 90, 95, 100, 105, 110, 115, 120, 125, 130, 135, 140, 145, or about 150 mg/40 g serving.

With this mind, it is contemplated to provide an amount of the resulting grains such that the composition contains AVA alone or in combination with GABA such that the AVA is provided in amounts from about 5 mg/40 g serving and up to about 40 mg/40 g serving to enhance relaxation, improve sleep and/or relaxation, reduce blood pressure, improve cardiovascular health, reduce stress, reduce oxidative stress, flow mediated dilation (FMD), reduce risk of developing hypertension, improve blood vessel function, and/or improve endothelial function.

To that end, a method of reducing oxidative stress includes providing a composition containing an amount of the resulting grains such that the composition includes AVA in amounts from about 1 mg/40 g serving to about 20 mg/40 g serving to reduce oxidative stress or from about 1, 2, 3, 4, 5, 6, 7, 8, 9, 10, 11, 12, 13, 14, 15, 16, 17, 18, 19, or about 20 mg/40 g serving. In other embodiments, a method of reducing one or more of FMD or blood pressure or of enhancing a feeling of sleepiness or decreasing the time duration to sleep includes providing a composition containing the resulting grains to provide AVA in amounts from about 5 mg/40 g serving to about 30 mg/40 g serving or from about 5, 6, 7, 8, 9, 10, 11, 12, 13, 14, 15, 16, 17, 18, 19, 20, 21, 22, 23, 24, 25, 26, 27, 28, 29, or about 30 mg/40 g serving.

### EXAMPLES

### Example 1

Oat seeds were provided and a sample was taken to measure the concentration of GABA and AVA in the provided oat seeds. Thereafter, the remaining oat seeds were sterilized for 10 minutes by application of a 0.1% NaOCl solution. Thereafter, the oats were steeped using DI water at a pH of 7.0 with a salinity and calcium content of zero. The steeping step was conducted at a temperature of 20° C. for 4 hours.

Thereafter, the steeped seeds were separated into three different batches to evaluate germination conditions. The first, referred to as “low temperature” (LT), subjected the steeped seeds to a germination temperature of 24° C. and relative humidity of at least 95% for 72 hours. The second, referred to as “heat shock” (HS), subjected the steeped seeds to the following germination conditions (each at a relative humidity of at least 95%): 42.8° C. for 15 hours, 32° C. for 3 hours, and 24° C. for 54 hours. The third, referred to as “high temperature” (HT), subjected the germinated seeds to a germination temperature of 42.8° C. and relative humidity of at least 95% for 72 hours.

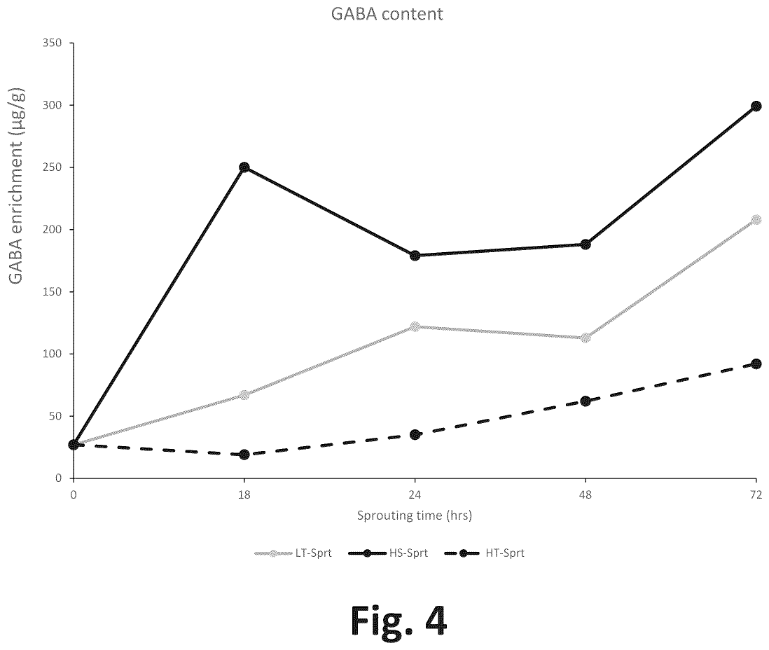

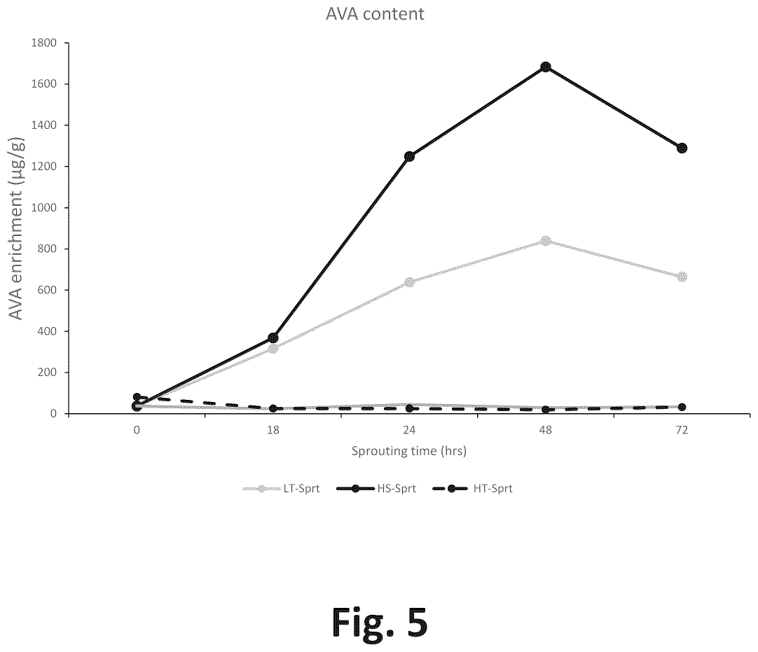

For each batch, samples of the germinated seeds were obtained at 18, 24, 28, and 72 hours and kilned at 74° C. for 6 hours. After kilning, the concentration of GABA and AVA for each sample was measured. The results are shown in FIGS. 4 and 5 with FIG. 4 showing the concentration of GABA over time and FIG. 5 showing the concentration of AVA over time.

### Example 2

Oat seeds were sterilized for 10 minutes by application of a 0.1% NaOCl solution. Thereafter, the oats were steeped using DI water at a pH of 7.0 with a salinity and calcium content of zero. The steeping step was conducted at a temperature of 20° C. for 4 hours.

The steeped seeds were separated into four separate groups and each group was subjected to conductive heat shock treatment using one of the following conditions: (a) 42° C. for 4.5 hours, (b) 42° C. for 9 hours, (c) 42° C. for 18 hours, and (d) 50° C. for 4.5 hours. After the tested heat shock step, the seeds were moved to a germination chamber where the seeds were germinated at 24° C. for 72 hours. Thereafter, the GABA content was measured.

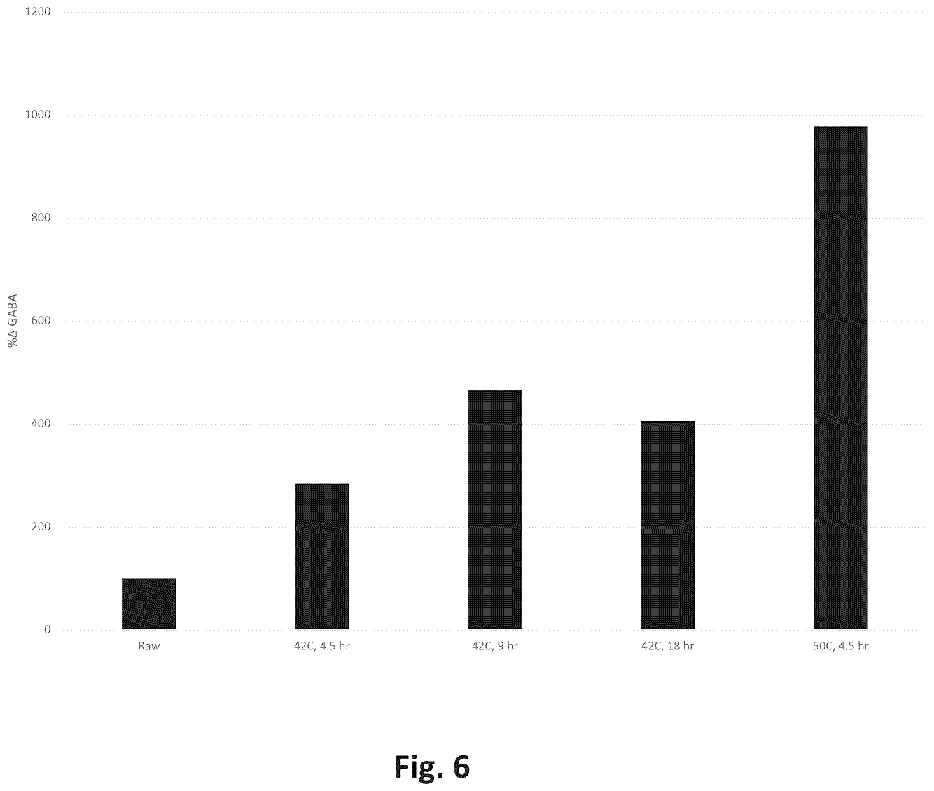

It was found that decreasing the 42° C. heat shock exposure time from 18 h to 9 h, increased % A GABA from 300% to 370%. Further decreasing the exposure time to 4.5 h was detrimental to the increase in GABA. However, the largest %Δ GABA occurred when the heat shock temperature was increased from 42° C. to 50° C. while keeping the exposure time to 4.5 h (880%). The results are shown in FIG. 6.

### Example 3

Oat seeds were sterilized for 10 minutes by application of a 0.1% NaOCl solution. Thereafter, the oats were steeped using DI water at a pH of 7.0 with a salinity and calcium content of zero. The steeping step was conducted at a temperature of 20° C. for 4 hours.

The steeped seeds were separated into nine separate groups and a heat shock treatment was conducted using microwave radiation under the following conditions: (1) 270 Watts for 10 seconds, (2) 270 Watts for 65 seconds, (3) 270 Watts for 120 seconds, (4) 510 Watts for 10 seconds, (5) 510 Watts for 65 seconds, (6) 510 Watts for 120 seconds, (7) 760 Watts for 10 seconds, (8) 760 Watts for 65 seconds, and (9) 760 Watts for 120 seconds. After the tested heat shock step, the seeds were moved to a germination chamber where the seeds were germinated at 24° C. for 72 hours. Thereafter, the GABA content was measured.

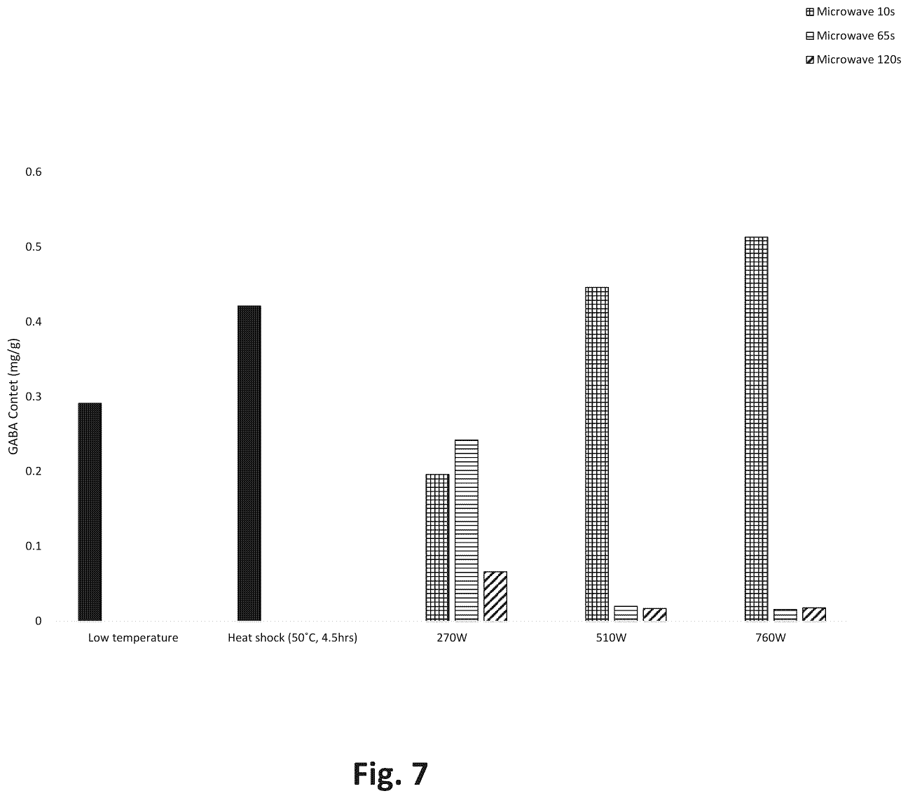

Heating the groats (the oat seeds) for 10 sec at 760 Watt gave the highest level of GABA. This treatment protocol produced 20% more GABA than conductive heat shock. Treatment with 510 Watts for 10 sec produced about the same level of GABA as a conductive heat shock treatment conducted at 50° C. for 4.5 hours. In addition, treatment with 760 Watts for 10 sec also produced a GABA content about the same or slightly greater than that achieved with a conductive heat shock treatment conducted at 50° C. for 4.5 hours. The results are shown in FIG. 7.

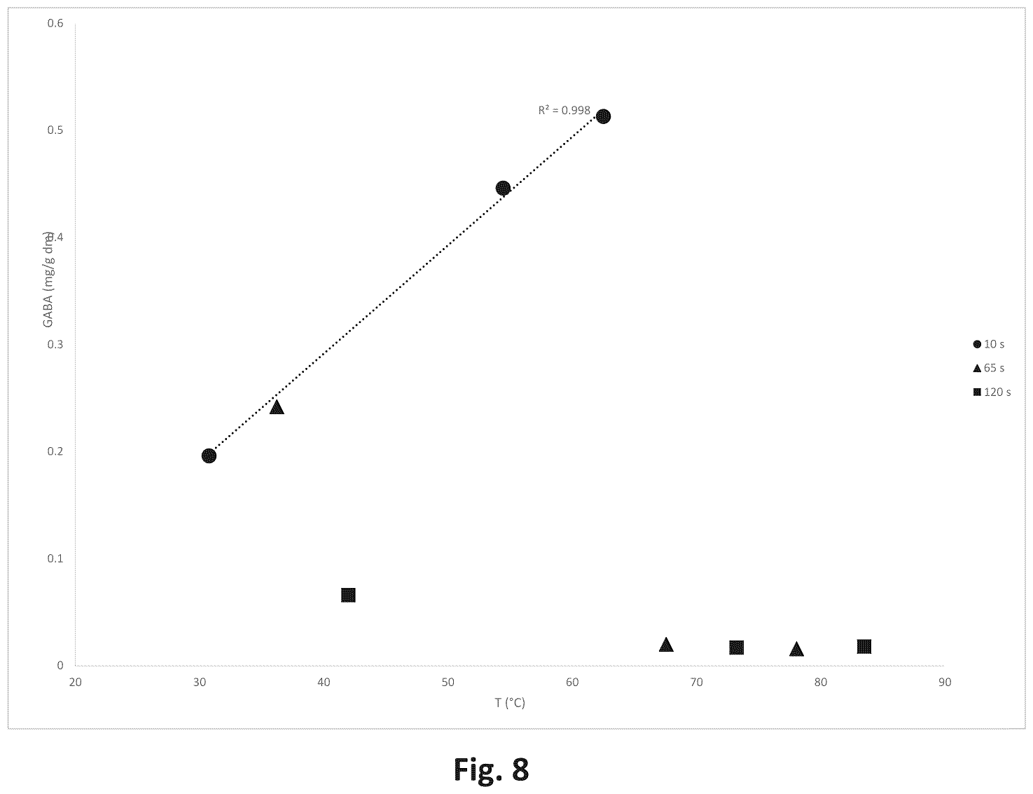

It was also observed that when the microwave treatment was short (10 seconds), there appeared to be a correlation between the GABA accumulation and the temperature at the surface of the oat groats, which is shown in FIG. 8.

While the concepts of the present disclosure are susceptible to various modifications and alternative forms, specific exemplary embodiments of the disclosure have been shown by way of example in the drawings. It should be understood, however, that there is no intent to limit the concepts of the present disclosure to the particular disclosed forms; the intention is to cover all modifications, equivalents, and alternatives falling within the spirit and scope of the invention as defined by the claims.

1. A method for increasing the concentration of one or more bio-active compounds in grains comprising:
steeping grain seeds for a period of time at a steeping temperature;
subjecting the steeped seeds to a heat shock step by subjecting the steeped seeds to a temperature higher than the steeping temperature for a period of time;
germinating the heat-shocked seeds by subjecting the heat-shocked seeds to a temperature less than the heat shock temperature for a period of time; and
kilning the germinated seeds.

2. The method of claim 1 wherein the grains are selected from oats, barley, and mixtures thereof.

3. The method of claim 1, wherein the steeping is conducted at a temperature between about 10° C. to about 30° C. for a period of time from about 2 hours to about 24 hours.

4. The method of claim 3 wherein the steeping includes wetting the grain seeds with an aqueous solution having a pH of about 7.0.

5. The method of claim 1 wherein the heat shock step is conducted using conductive heating or microwave radiation.

6. The method of claim 5 wherein the heat shock step is conducted using conductive heating with a temperature in the range of about 25° C. to about 65° C.

7. The method of claim 6 wherein the heat shock step period of time is between about 0.25 hours to about 18 hours.

8. The method of claim 5 wherein the heat shock step is conducted using microwave radiation with a power to achieve a surface temperature on the grain seed surface between 30° C. and 65° C.

9. The method of claim 8 wherein the heat shock step period of time is between about 10 seconds to about 120 seconds.

10. The method of claim 1 wherein the germination is conducted at a temperature between about 20° C. and about 40° C.

11. The method of claim 1 wherein the germination is conducted for a period of time between about 18 hours and 96 hours.

12. The method of claim 1 kilning is initiated when a size of a radicle is no more than 90% of a size of the seed.

13. The method of claim 12 wherein kilning is conducted at a temperature and for a period time so that the kilned seeds have a moisture content between about 2% and about 12%.

14. The method of claim 1 further comprising further processing the kilned seeds by flaking or milling.

15. The method of claim 1 wherein the kilned seeds have a concentration of one or more bio-actives that is at least 120% greater than the same one or more bio-actives in the raw unprocessed grain seeds.

16. The method of claim 15 wherein the one or more bio-actives includes gamma aminobutyric acid (GABA) or Avenanthramide (AVA).

17. The method of claim 1 wherein
the steeping includes wetting the grain seeds with an aqueous solution having a pH of about 7.0, at a temperature between about 10° C. to about 30° C. and for a period of time from about 2 hours to about 24 hours;
the heat shock step is conducted using conductive heating at a temperature in the range of about 25° C. to about 65° C. for a period of time between about 0.25 hours to about 18 hours or is conducted using microwave radiation with a power to achieve a surface temperature on the grain seed surface between 30° C. and 65° C. for a period of time between about 10 seconds to about 120 seconds;
the germination is conducted at a temperature between about 20° C. and about ° C. for a period of time between about 18 hours and 96 hours; and
the kilning is initiated when a size of a radicle is no more than 90% of a size of the seed and is conducted at a temperature and for a period time so that the kilned seeds have a moisture content between about 2% and about 12%.

18. Sprouted oats having a concentration of GABA that is at least 120% greater than a concentration of GABA in a raw oat seed of the same variety or a concentration of AVA that is at least 120% greater than a concentration of AVA in a raw oat seed of the same variety.

19. A method for modulating energy and mood attributes in a mammal comprising providing a composition containing an effective amount of the sprouted oats of claim 18.
